(19) United States
(12) Patent Application Publication
(10) Pub. No.: US 2011/0059182 A1
(43) Pub. Date: Mar. 10, 2011

(54) SPRAYING DEVICE AND RELATED METHOD FOR CELL AGGREGATES AND CELL AGGREGATE SUSPENSION THEREOF

(75) Inventors: Shayn Peirce Cottler, Charlottesville, VA (US); Adam J. Katz, Charlottesville, VA (US); Peter Stapor, New Orleans, LA (US); Peter J. Amos, Charlottesville, VA (US)

(73) Assignee: University of Virginia Patent Foundation, Charlottesville, VA (US)

(21) Appl. No.: 12/866,200

(22) PCT Filed: Feb. 5, 2009
(86) PCT No.: PCT/US09/33220

§ 371 Date: Nov. 24, 2010

Related U.S. Application Data
(60) Provisional application No. 61/026,318, filed on Feb. 5, 2008.

(51) Int. Cl. A61K 9/16; A61K 35/12; A61P 17/02; A61P 43/00; A61P 23/00; A61M 35/00; A61M 5/00

(52) U.S. Cl. 424/499; 424/93.7; 604/310; 604/73

(57) ABSTRACT

A sprayer device and related method that delivers multicellular aggregates in suspension. A purpose of the sprayer embodiment may be to deliver cells into chronic wound beds, and additional purposes of the sprayer include additional application in other cell delivery environments, such as during open-heart surgery or other surgeries where it may be desirable to treat internal organs with aggregates of stem cells.

### RELATED APPLICATIONS

The present invention claims priority from U.S. Provisional Application Ser. No. 61/026,318 filed Feb. 5, 2008, entitled “Spraying Device for Cell Aggregate and Related Method thereof;” the disclosure of which is hereby incorporated by reference herein in its entirety.

This application is related to PCT International Application No. PCT/US2007/021432, filed Oct. 5, 2007 to Katz, et al., entitled “Methods and Compositions Useful for Diabetic Wound Healing.” the disclosures of which is hereby incorporated by reference herein in its entirety.

### BACKGROUND OF THE INVENTION

Chronic wounds, such as diabetic wounds, are a major global health and economic burden. It is estimated that there are 10.8 million chronic wounds in the United States annually, and of these, diabetic wounds account for almost twenty percent (de Zoysa et al., 2005). Approximately six percent of diabetic patients have a chronic wound at any point in time, and 50-70 percent of diabetic wounds require adjuvant therapies. However, many such wounds fail to heal despite adequate implementation of fundamental treatment strategies, including debridement, various topical agents, protective dressings, and avoidance of pressure. In the recent past, various biological wound therapies (“advanced wound therapeutics”) have become available to the clinician. However, from a clinical standpoint, these products have not been universally embraced: mechanisms are not clearly understood, efficacy not always predictable or reproducible, and treatment guidelines are undefined. From a commercial standpoint, high manufacturing costs, inefficient product shelf-life/stability, and burdensome product use/application remain major barriers to acceptance and use of these products. This is reflected by the fact that there is no clear market leader. Given the significant expense of these products and the fact that their (beneficial) impact on a wound is not assured, many clinicians are skeptical of their overall cost-effectiveness, and therefore their role in the care of wounds. For all of these reasons, commercial opportunity still exists in this competitive but lucrative field and a long felt need for improved wound care.

### BRIEF SUMMARY OF INVENTION

Human adipose-derived cells (HACs) represent a novel cell platform on which to base a new generation of wound biologics and regenerative therapies. It should be appreciated that human adipose-derived cells may include, but are not limited thereto the following: Human adipose stem cells (HASCs), human adipose progenitor cells (HAPCs), human adipose endothelial (HAECs), and human adipose stromal cells (HAStrCs) or any combination thereof. Any of the aforementioned cell (HASC, HASC, HAPC, HAEC) may be autologous or non-autologous. HACs have a number of attributes that make them an appealing and unique therapeutic strategy for tissue regeneration and healing including, but not limited thereto the following:
hACs are available and safely accessible via liposuction procedures in adult patients,
hACs exhibit tremendous regenerative and therapeutic potential when delivered exogenously in an array of different injury and disease states, both in humans and in animal models,
Safety of injecting hACs in humans have recently been substantiated in clinical trials/cases,
hACs are available in large quantities,
harvest of hACs results in no major donor site defect or morbidity; rather, it is appealing to most donors/patients.

Referring to PCT International Application No. PCT/US2007/021432, filed Oct. 5, 2007 to Katz, et al., entitled “Methods and Compositions Useful for Diabetic Wound Healing,” applicant has recently developed a novel patent-pending therapy for healing chronic wounds that uses hASCs as the building blocks for 3-dimensional constructs of lipo-derived cells, know as Self-organizing Niche Milieus (SNiM). For the purpose of this disclosure the SNiM shall also be referred to as multicellular aggregates, which shall be abbreviated “MA”. MA offer significant potential advantages related to cost-efficient commercialization, therapeutic efficacy, and end-user appeal. Applicant has conducted extensive pre-clinical trials in an established murine model of delayed diabetic wound healing (db/db mice full-thickness excisional wounds), and the data shows that SNiMs (or MAs) significantly expedite the healing process and cause no adverse effects, such as inflammation, immune rejection, or tumorigenesis. Moreover, SNiM therapy (or MA therapy) accelerates the healing of mouse diabetic wounds to the extent that the rate of healing is indistinguishable from healthy, non-diabetic control animals. In other words, SNiM therapy (or MA therapy) allows diabetic wounds to heal like normal healthy wounds, and if this level of benefit (or anything approaching this level of benefit, for that matter) could be achieved in humans it would have tremendous impact on patient morbidity and quality of life.

An aspect of an embodiment or partial embodiment provides a novel delivery device, a “SNiM Sprayer” (“MA Sprayer”), which can deliver SNiMs (cell aggregates or MA) into wounds (or intended target or site).

An aspect of an embodiment or partial embodiment of the present invention (or combinations of various embodiments in whole or in part of the present invention) comprises a sprayer device and related method (and related suspension thereof) to deliver cell aggregates into wound beds (or other targets of the subject such as other tissues and organs of interest, such as, but not limited thereto, the epicardium during open-heart surgery).

Commercially available cell-based (biologic) wound care products (e.g. Epicel, Laserskin, Celaderm, Transcyte, Dermagraft, Apligraf, Orcel) are typically fabricated as, and delivered into skin wounds as sheets (or constructs) of engineered/processed substrate (e.g. silicone, cadaver skin, nylon, porcine small intestinal mucosa, bovine-derived collagen). While this method of delivery has proven useful for many products, they are associated with time, manufacturing, storage/inventory, shipping, cost and end-user constraints and limitations. In contrast, our pre-clinical data suggests that SNiM therapy (or MA therapy) facilitates and enables a unique delivery approach and therapeutic strategy that minimizes the shortcomings described for the aforementioned conventional techniques.

The various embodiments of the present invention or partial embodiments provide advantages and improved features and characteristics compared to other conventional designs. For instance, an embodiment or an aspect of an embodiment delivers aggregates of cells rather than on a cellular level. An analogy, for example, may be that an embodiment of the present invention delivers “snowballs” rather than “snowflakes” of the conventional method. For instance, the applicant's preclinical data and related studies demonstrate a biological/potency advantage of the “snowballs” over the “snowflakes.” Further, another distinction is that an embodiment or an aspect of an embodiment of the present invention delivers adipose derived aggregates.

In an exemplary embodiment prototype the device is capable of spraying approximately 1 mm diameter hASC-SNiMs (or MA) into wound beds. An aspect of various embodiments of the present invention (or partial embodiment of the present invention) may also be extended to deliver cell aggregates composed of cell types other than hASCs (including bone marrow-derived stem cells, etc.), in addition to hASCs. It may be noted that an aspect of an embodiment (or partial embodiment) of the sprayer differ from other cell sprayers that have previously been developed. The present invention embodiment sprayer (or partial embodiment) delivers cell aggregates; not individual cells in suspension, as has been accomplished with conventional approaches.

Another aspect of an embodiment or partial embodiment can be further differentiated (exemplified) by the concept of modular niche therapy (SNiMs/MAs) as compared to cell suspension therapy. An embodiment of the present invention for the delivery of the SNiMs/modular approach or technology includes delivery of established cell-cell contacts/signaling, ECM (extracellular matrix) components, and numerous bioactive factors produced and sequestered by such. Whereas conventional approaches may provide delivery of single cell suspensions.

Regarding, for example, formation of multicellular aggregates (MA), see co-owned PCT International Application No. PCT/US2007/002572, filed on Jan. 30, 2007 to Khurgel, et al., entitled “Methods of Preparing and Characterizing Mesenchymal Stem Cell Aggregates and Uses Thereof” and co-owned PCT International Application No. PCT/US2007/021432, filed Oct. 5, 2007 to Katz, et al., entitled “Methods and Compositions Useful for Diabetic Wound Healing.” the disclosures of which are hereby incorporated by reference herein in their entirety. Applicant demonstrates that ASCs cultured (i.e. formulated) as 3D MAs secrete more growth factors, more matrix components, more in vivo potency, and have more reproducible gene expression pattern than the same cells cultured as 2-D monolayers and delivered as single cell suspensions.

An aspect of various embodiments of the present invention (or partial embodiment) may provide a number of novel and nonobvious features, elements and characteristics, such as but not limited thereto, the following: a sprayer that delivers cell aggregates (instead of individual cells in suspension). A purpose of the sprayer embodiment may be to deliver cells into chronic wound beds, and additional purposes of the sprayer include additional application in other cell delivery environments, such as during open-heart surgery or other surgeries where it may be desirable to treat internal organs with aggregates of stem cells.

An aspect of various embodiments (or partial embodiments) of the present invention comprises, but not limited thereto, the following: a sprayer device that includes a funnel-shaped cone shield adaptation that 1) (physically restrains the spray/cells to the intended target), and 2) regulates the distance at which the sprayer is held while the cell aggregates are delivered to the wound bed (or other tissue/organ of interest). This cone feature, therefore, not only enhances the safety of using the device (by physically containing the sprayed biohazardous cell aggregates), but it also facilitates more reproducible and controlled delivery of the cell aggregates into the wound bed by regulating the distance at which the sprayer is held from the wound bed, thus minimizing the potential for operator error.

An aspect of an embodiment (or partial embodiment) comprises a method of delivering one or more multicellular aggregates to a target surface of a subject, wherein the target surface may comprise at least one of a wound, tissue, or organ, etc. The method may comprise: obtaining the multicellular aggregates (MA); suspending the multicellular aggregates (MA); and spraying the suspension of multi-cellular aggregates (MA) on the target surface.

An aspect of an embodiment (or partial embodiment) comprises a device for delivering one or more multicellular aggregates to a target surface of a subject. The target surface may comprise at least one of a wound, tissue, or organ, etc. The device may comprise: a multicellular aggregate (MA) source; a multicellular aggregate (MA) tube (or the like) for accommodating a suspension of the multicellular aggregates (MA) traveling from the proximal end to the distal end of the multicellular aggregate tube; an air flow tube (or the like), whereby the air flow tube is enabled for accommodating the air flow traveling from the proximal end to the distal end of the air flow tube; and a nozzle (or the like), whereby the air flow exits the nozzle, and whereby the air flow carries the multicellular aggregates (MA) suspension for deposition on the target surface.

An aspect of an embodiment (or partial embodiment) comprises a delivered deposit on at least a portion of a target surface of a subject, whereby the target surface comprises at least one of a wound, tissues, or organs, etc. The deposit may comprise a biological dressing, network, association, slurry, gel, or system of a multicellular aggregates (MA).

These and other objects, along with advantages and features of the invention disclosed herein, will be made more apparent from the description, drawings and claims that follow.

### BRIEF DESCRIPTION OF THE DRAWINGS

The foregoing and other objects, features and advantages of the present invention, as well as the invention itself, will be more fully understood from the following description of preferred embodiments, when read together with the accompanying drawings

The accompanying drawings, which are incorporated into and form a part of the instant specification, illustrate several aspects and embodiments of the present invention and, together with the description herein, serve to explain the principles of the invention. The drawings are provided only for the purpose of illustrating select embodiments of the invention and are not to be construed as limiting the invention.

FIG. 1(A) provides a schematic elevation view of an embodiment of the sprayer device. FIG. 1(B) provides schematic partial longitudinal view of FIG. 1(A).

FIGS. 2(A)-(B) provide photographic depictions of components of the sprayer device disassembled and the sprayer device assembled, respectively.

FIG. 3 provides photographic depiction of the sprayer device with a cone attachment (deflector or regulator).

FIGS. 4(A)-(B) provide photographic depictions of a sprayer undergoing various testing protocols inside the biosafety hood of the Applicant's laboratory. It may be noted that the fluid containing the SNiMs (MA) is fed into the sprayer using an air pump (FIG. 4(A)).

FIG. 5(A) provide photographic depictions of proof of concept spray created by spraying cell culture media (or fluid) alone (in the absence of hASC-SNiMs (or MA)) that shows the droplet size that can be delivered using the sprayer device. FIG. 5(B) provides photographic depiction of a close-up of cell culture media droplets sprayed with the sprayer device (no hASC-SNiMs (or MA)).

FIGS. 6(A)-(B) provide photographic depictions of an example hASC-SNiM (or MA) that was sprayed onto tissue culture plastic and allowed to adhere. The hASC cells surrounding the spheroid-shaped hASC-SNiMs (or MA) indicates that the sprayed hASC-SNiMs are still viable (i.e. alive) after spraying, and able to generate proliferative hASCs from it. These results support that the spraying of hASC-SNiMs (or MA) does not negatively impact their functionality. FIG. 6(A) was taken with a light microscope of 10×. FIG. 6(B) is a higher magnification of the same hASC-SNiM (or MA) of FIG. 6(A), taken with a light microscope of 20×.

FIG. 7(A) provides a schematic elevation view of an embodiment of the sprayer device. FIG. 7(B) provides schematic partial view of FIG. 7(A).

FIG. 8 provides a schematic elevation view of an embodiment of the sprayer device.

### DETAILED DESCRIPTION OF THE INVENTION

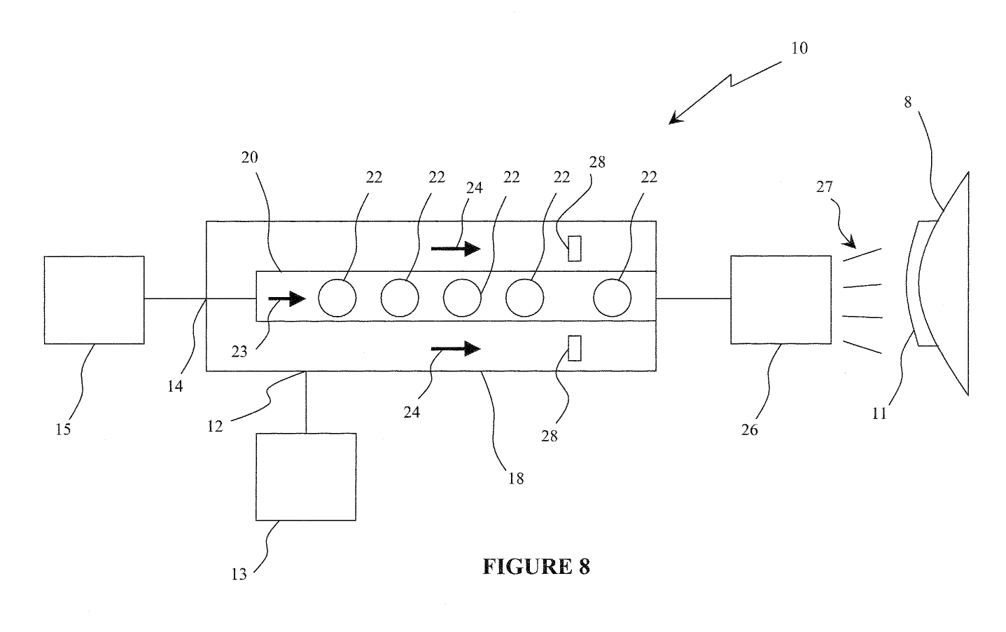

Referring to FIG. 8, an aspect of an embodiment (or partial embodiment) may comprise, but not limited thereto, a sprayer device 10 for delivering multicellular aggregates 22 into or on targets 8 of interest (e.g., tissues, organs, chronic wound beds, or other designated targets). The sprayer device 10 may include two or more inlet ports. One port may be an air port 12 where the air flow enters and another port may be a multicellular aggregate port 14 whereby the multicellular aggregates (MA) 22 (delivered in a fluid suspension or vehicle) enters. The air port 12 may be in communication with the air source 13 and other components and the multicellular aggregate port 14 may be in communication with the aggregate and/or suspension/fluid (vehicle) source 15 or other components. It should be appreciated that other components and systems may be in communication with the sprayer device 10 within the context of an embodiments of the invention. The sprayer device 10 has an outer tube 18 or other like conduit and an inner tube 20 or other like conduit. The multicellular aggregates (MA) 22 flow, as indicated by arrow 23, through the inner tube 20 onward to the proximal end of the sprayer device 10 (i.e., the end that is held closest to the wound bed or tissue/organ of interest (or other designated or desired target) where the multicellular aggregates (MA) 22 are to be delivered. The air may flow 24 around the inner tube 20 down through an outer tube 18. As the air flow 24 approaches the nozzle 26 at the proximal end, it flows through the pores 28, apertures, vents, deflections, so as to effect or regulated the air flow. The air flow 24 encounters or mixes with the flow of the multicellular aggregates (MA) 22 (which may exist as a combination of fluid and cells, i.e., fluid suspension,) at or proximal to the nozzle 26. As the air flow 24 exits the nozzle it effectively “sprays” the fluid and multicellular aggregates 24 (i.e., mixture) exiting the small-diameter of the inner tube 20.

Still referring to FIG. 8, the spray 27 produces a dispersed biological dressing, network, association, slurry, gel, or system of MAs 11 on and/or in the target of interest 8. The MAs as discussed herein may be suspended in a variety of media, fluids, or vehicles, etc. Although not shown, a deflector, such as a cone or other surface, aperture, etc., may be used to shield or redirect the flow of the MA or air inside and/or outside the inner and outer tubes, or nozzles.

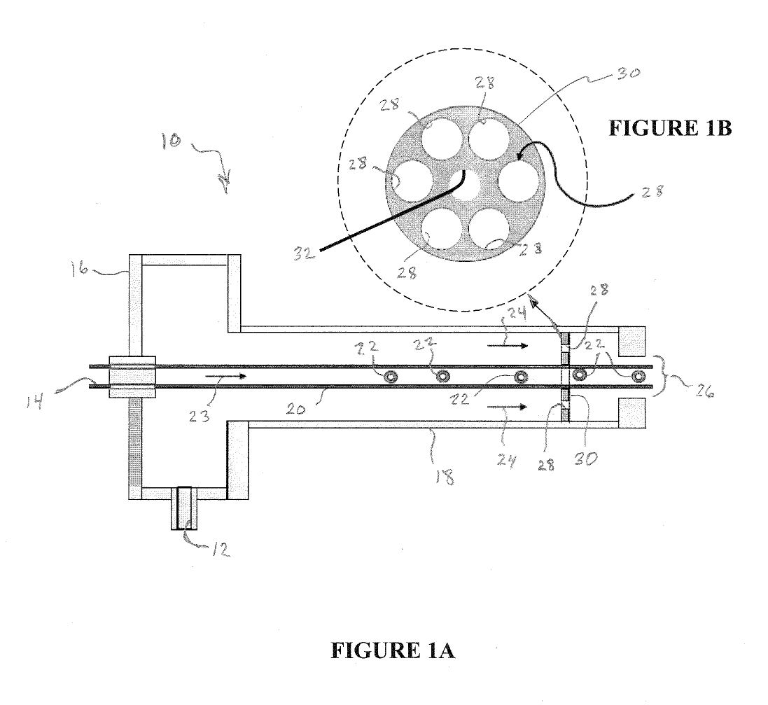

Referring to FIG. 1, an aspect of an embodiments (or partial embodiments) of the present invention comprises, but not limited thereto, a sprayer device 10 for delivering cell aggregates into tissues of interest (e.g. chronic wound beds, or other designated targets). The sprayer device 10 may include two or more inlet ports that feed a main manifold 16. One port may be an air port 12 where the air flow enters and another port may be a cell aggregate port 14 where the multicellular aggregates 22 (delivered in a fluid suspension) enters. It should be appreciated that the manifold may be avoided or replaced with a similar component so long as the air port 12 and cell aggregate port 14 can be in communication with the sprayer device 10. The sprayer device 10 has an outer tube 18 or other like conduit and an inner tube 20 or other like conduit. The multicellular aggregates 22 flow, as indicated by arrow 23, through the inner tube 20 onward to the proximal end of the sprayer device 10 (i.e., the end that is held closest to the wound bed or tissue/organ of interest (or other designated or desired target) where the multicellular aggregates 22 are to be delivered. The air flows 23 around the inner tube 20 down through an outer tube 18. As the air flow 24 approaches the nozzle 26 at the proximal end, it flows through the pores 28 in a support 30 or suitable structure for providing pores or apertures to affect the air flow (see FIG. 1(B)). The air flow 24 encounters or mixes with the flow of the multicellular aggregates 22 (which may exist as a combination of fluid and cells, i.e., fluid suspension) at or proximal to the nozzle 26. As the air flow 24 exits the nozzle it effectively “sprays” or disperses the fluid and multicellular aggregates 24 (i.e., mixture) exiting the small-diameter of the inner tube 20. As shown in FIG. 1(B), this example embodiment illustrates the inner tube 20 passing through a tube aperture 32 of the support 30. It should be appreciated that the inner tube 20 can pass through the support or adjacent to the support depending on the particular design or approach.

It should be appreciated that there may a variety of potential delivery vehicles (fluid, fluid suspension, media) or portions of the delivery vehicle for spraying/delivering the MAs may include, but are not limited thereto, the following: biologic and synthetic biocompatible systems such as reverse-thermal gelling poloxamers (e.g. Pluronic F68, Pluronic F127), chitosan, hyaluronic acid, hydrogels, buffers, saline, thrombin/fibrin, platelet rich plasma (PRP), etc., or any combinations thereof. For example, but not limited thereto, refer to PCT International Application No. PCT/US2007/021432, filed Oct. 5, 2007 to Katz, et al., entitled “Methods and Compositions Useful for Diabetic Wound Healing,” and PCT International Application No. PCT/US2007/001896, filed on Jan. 25, 2007 to Laurencin, et al., entitled “Methods for Regulating Gelation of Polysaccharide Solutions and Uses Thereof” the disclosures of which are hereby incorporated by reference herein in their entirety.

It should be appreciated that there may be a variety of potential delivery vehicles (fluid, fluid suspension, media) or portions of the delivery vehicle for spraying/delivering the MAs that may include, but not limited thereto, the following: medication, drug, coloration, dye, (bioactive factor (e.g. growth factor, cytokine, hormone)) or anesthetic, etc., or any combination thereof.

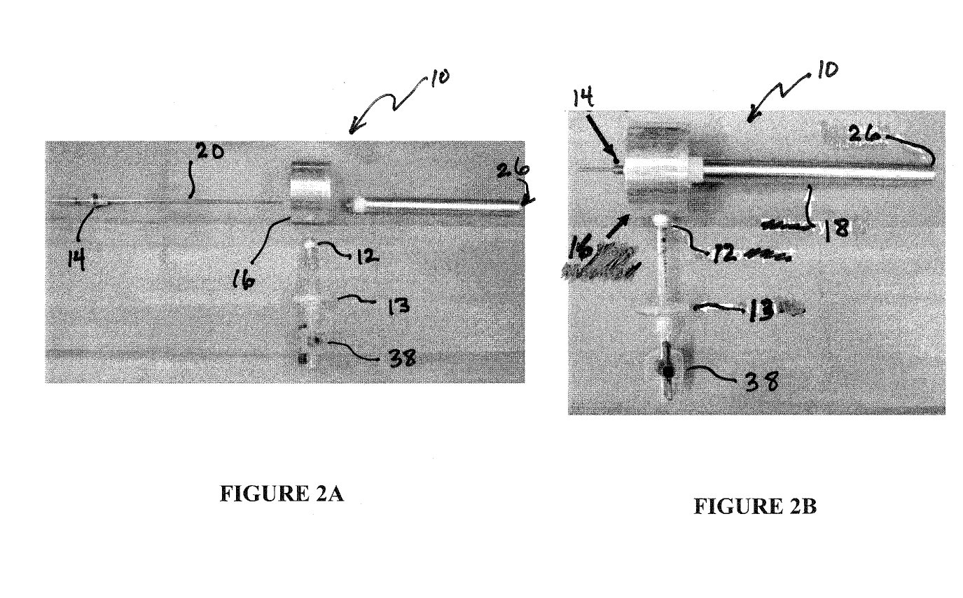

Turning to FIG. 2, FIGS. 2(A)-(B) provide photographic depictions of components of the sprayer device 10 disassembled and the sprayer device assembled, respectively. The sprayer device 10 illustrates a non-limiting embodiment comprising the manifold 16, air port 12, multicellular aggregate port 14, outer tube 18, nozzle 26 and air filter 13, which may be used to sterilize the incoming air before entering the air flow port 12. Also shown is an adapter 38, such as a Luer adapter or other applicable connection or adapters.

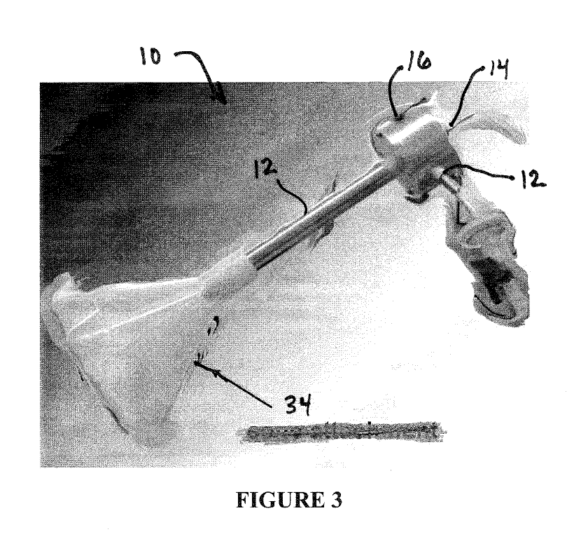

Turning to FIG. 3, FIG. 3 illustrates the sprayer device 10 as similarly shown in FIGS. 1-2 and as discussed throughout this disclosure with the addition of a cone 34 or other suitable conical device or other optimum-contoured shaped device that is desired or required. The cone 34 may function to shield the flow field and regulate the distance between the nozzle and the tissue bed of interest (or other desired or required target).

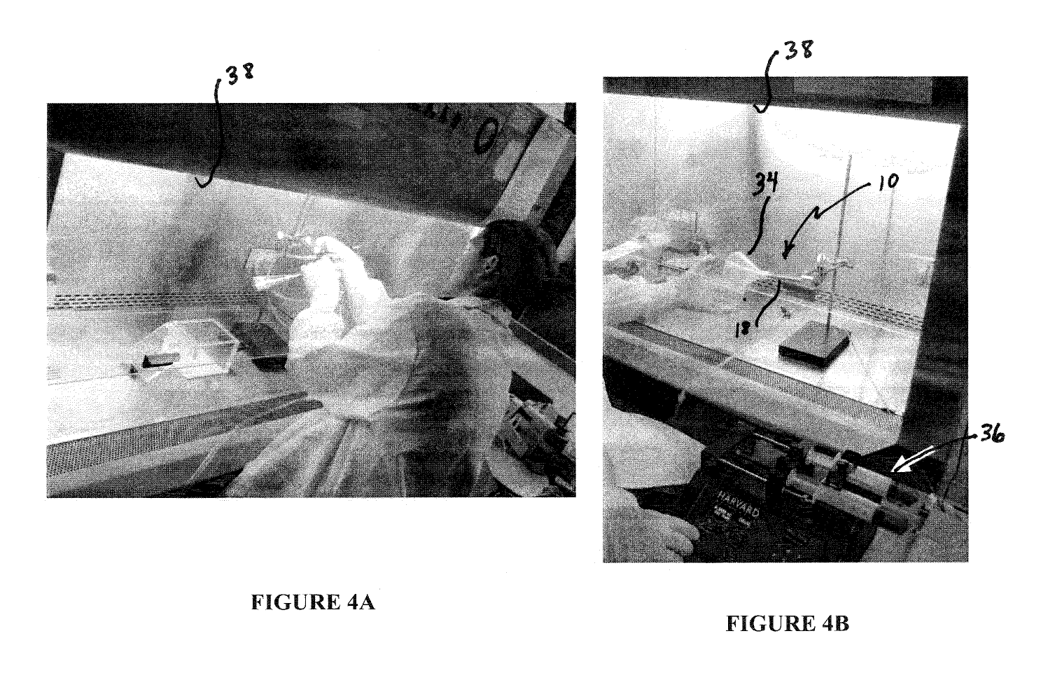

FIGS. 4(A)-(B) provide photographic depictions of a sprayer undergoing various testing protocols inside the biosafety hood 38 of the Applicant's laboratory. It may be noted that the fluid containing the SNiMs (MA) is fed into the sprayer using pump 36, such as Double Harvard Apparatus Syringe Pump (FIG. 4(B)).

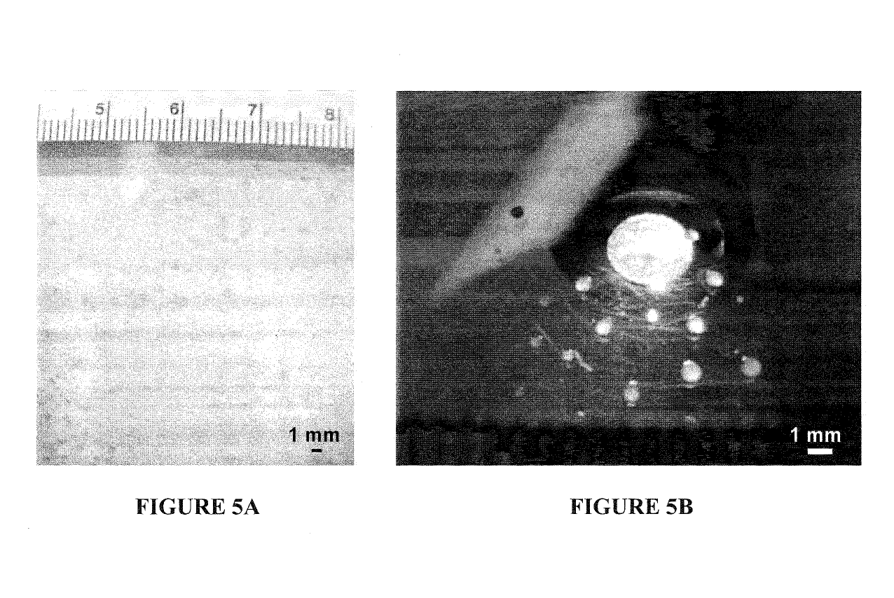

Turing to FIG. 5, FIG. 5(A) provide photographic depictions of proof of concept spray created by spraying cell culture media alone (in the absence of hASC-SNiMs (or MA)) that shows the droplet size that can be delivered using the sprayer device. FIG. 5(B) provides photographic depiction of a close-up of cell culture media droplets sprayed with the sprayer device (no hASC-SNiMs (or MA)). This proof of concept illustrates that a medium (in this case cell media) can be sprayed as droplets. This aspect of the embodiment illustrates the delivery of cell aggregates, which can range from about 250 μm to about 2 mm in diameter/length, typically about 250 μm to about 800 μm.

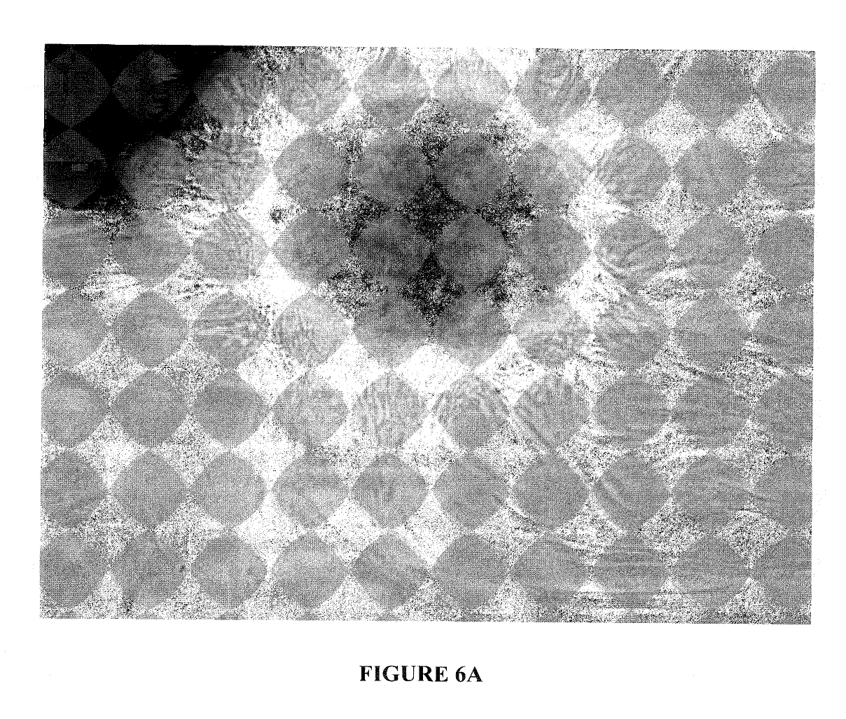

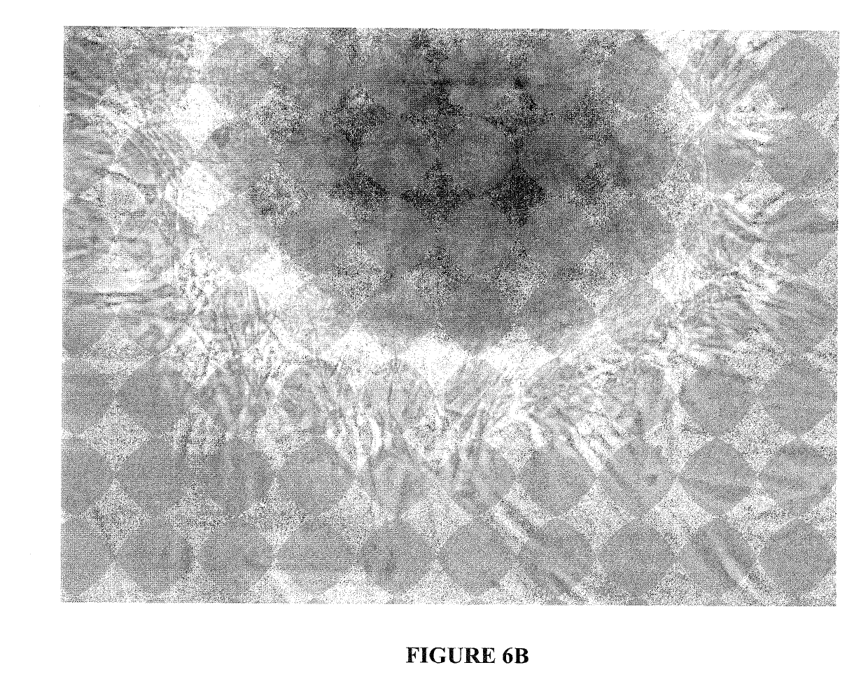

FIGS. 6(A)-(B) provide photographic depictions of an example hASC-SNiM (or MA) that was sprayed onto tissue culture plastic and allowed to adhere. The hASC cells surrounding the spheroid-shaped hASC-SNiMs (or MA) suggest that the sprayed hASC-SNiMs are still viable (i.e. alive) after spraying, and able to proliferate hASCs from it. These results support that the spraying of hASC-SNiMs (or MA) does not negatively impact their functionality. FIG. 6(A) was taken with a light microscope of 10×. FIG. 6(B) is a higher magnification of the same hASC-SNiM (or MA) of FIG. 6(A, taken with a light microscope of 20×.

The materials are not limited to stainless steel; it is possible to have a one-time use, disposable device made of, for example, plastic or polymers (or other materials and compositions as desired or required). It should be appreciated that various sizes, dimensions, contours, rigidity, shapes, flexibility and materials of any of the embodiments discussed throughout may be varied and utilized as desired or required.

An aspect of an embodiment (or partial embodiment) may comprise a method and/or related device/system for delivering one or more multicellular aggregates to a target surface of a subject, wherein the target surface may comprise at least one of a wound, tissue, or organ, etc. The method may comprise: obtaining the multicellular aggregates (MA); suspending the multicellular aggregates (MA); and spraying the suspension of multi-cellular aggregates (MA) on the target surface. The process of obtaining may include fabricating the multicellular aggregates by assembling cells into 3-D aggregates that have already grown in culture. The process of obtaining may include fabricating the multicellular aggregates by pretreating cells and/or pretreating multicellular aggregates (MA) that have already grown in culture.

At least one of the multicellular aggregates (MA) may have a cross-section that includes the following ranges: about 100 μm to about 10 mm; about 200 μm to about 5 mm; about 250 μm to about 2 mm; about 250 μm to about 800 μm; greater than about 10 mm, or less than about 100 μm, or any combination thereof for the various MAs. It should be appreciated that the cross-section of any of the multicellular aggregate (MA) may vary as required or desired for any particular application of the method, devices, and/or deposits disclosed, discussed or referenced herein.

The suspension of multicellular aggregates (MA) may comprise: human adipose-derived cells (hACs). The human adipose-derived cells (hACs) may be are autologous, allogeneic, or any combination thereof.

The human adipose-derived cells (HACs) may comprise at least one of: Human adipose stem cells (HASCs), human adipose progenitor cells (HAPCs), human adipose endothelial (HAECs), or human adipose stromal cells (HAStrCs), or any combination thereof.

The suspension of multicellular aggregates (MA) may comprise aggregates comprising at least one of: bone marrow-derived stem cells, keratinocytes, fibroblasts, hematopoietic stem cells, endothelial cell progenitor cells, perivascular (pericyte) progenitor cells, or any other type of tissue repair cell, stem cell, progenitor cell, pluripotent cell, or embryonic stem cells, etc. or any combinations thereof.

The suspension of multicellular aggregates (MA) may be homogeneous. Homogeneous indicates that within a multicellular aggregate (MA) the cell types are the same. The suspension of multicellular aggregates (MA) may be a hybrid type. Hybrid indicates that within a multicellular aggregate (MA) the cell types of two or more cells are different. The suspension of multicellular aggregates (MA) may comprise two or more types of aggregates. For example, at least one multicellular aggregate (MA) is different compared to at least another multicellular aggregate (MA).

The wound, for example, may comprise at least one of pressure ulcer, venous stasis ulcer, diabetic ulcer, burn, surgical or traumatic wound, etc. or any combination thereof.

The tissue, for example, may comprises at least one of epicardium, endocardium, vascularized tissue, dermis, epidermis, hypodermis (subcutaneous), skeletal muscle, adipose, nervous tissue, fascia, or bone, etc., or any combination thereof.

The organ, for example, may comprises at least one of skin, heart, lung, brain, uterus, colon, eye, kidney, liver, stomach, small bowel, or pancreas, etc.

The number of the multicellular aggregates (MA) per unit volume of the suspension of multicellular aggregates (MA) may comprise a variety of ranges including the following: about 1 to about 10,000 multicellular aggregates per ml; about 1 to about 5,000 multicellular aggregates per ml; about 1 to about 2,000 multicellular aggregates per ml; greater than about 10,000 multicellular aggregates per ml; or less than about 1 multicellular aggregate per ml, or any combination thereof. It should be appreciated that the number of the multicellular aggregate (MA) per unit volume of the suspension may vary as required or desired for any particular application of the method, devices, and/or deposits disclosed, discussed or referenced herein.

The multicellular aggregates (MA) suspension may comprise a variety of ranges of volume including the following: about 0.1 ml to about 10,000 ml; about 1 ml to about 5,000 ml; about 1 ml to about 1,000 ml; about 1 ml to about 500 ml; greater than about 10,000 ml; less than about 0.1 ml, or any combination thereof. It should be appreciated that the ranges of volume of the multicellular aggregate (MA) suspension may vary as required or desired for any particular application of the method, devices, and/or deposits disclosed, discussed or referenced herein.

The multicellular aggregates (MA) suspension spray may have a flow rate in a variety of ranges including the following: about 0.05 ml/min to about 5000 ml/min; about 0.05 ml/min to about 1000 ml/min; about 0.1 ml/min to about 500 ml/min; greater than about 5000 ml/min; or less than about 0.05 ml/min, or any combination thereof. It should be appreciated that the flow rate of the multicellular aggregate (MA) suspension may vary as required or desired for any particular application of the method, devices, and/or deposits disclosed, discussed or referenced herein.

The delivering may comprises adherence of MAs to at least a portion of the target surface as desired or required.

The deposited suspension of multi-cellular aggregates (MA) produces a dispersed biological dressing, network, association, slurry, gel, or system of MAs, or any combination thereof on at least a portion of the target surface. The dispersion on at least a portion of the target surface promotes bioactive factors. The bioactive factors may comprises at least one of VEGF, HGF, or TGF-beta, etc. or any combination thereof. The dispersion on at least a portion of the target surface promotes matrix factors. The matrix factors may comprise at least one of collagen, fibronectin, decorin, or tenascin C, etc., or any combination thereof. The dispersion on at least a portion of the target surface comprises cells that promote subsequent cell-cell interactions. The dispersion on at least a portion of the target surface promotes bioactive factors, matrix factors, or cell-cell interactions, etc., or any combination thereof.

The suspension comprises a fluid or media may comprise at least one of the following: biologic and synthetic biocompatible systems such as reverse-thermal gelling poloxamers (e.g. Pluronic F68, Pluronic F127), chitosan, hyaluronic acid, hydrogels, buffers, saline, thrombin/fibrin, or platelet rich plasma (PRP), etc. or any combinations thereof.

The suspension may comprises a fluid or media comprising at least one of the following: medication, drug, coloration, dye, bioactive factor, or anesthetic, etc. or any combination thereof.

It should be appreciated that a multicellular aggregate (MA) comprises two or more cells. In an embodiment the number of cells in a multicellular aggregate (MA) includes the range of about 25,000 cells to about 50,000 cells. It should be appreciated that the number of cells in a multicellular aggregate (MA) may be greater than about 50,000 cells. It should be appreciated that the number of cells in a multicellular aggregate (MA) may be greater than about 200,000 cells. It should be appreciated that the number of cells in a multicellular aggregate (MA) may be greater than about 500,000 cells. It should be appreciated that the number of cells in a multicellular aggregate (MA) may be less than about 25,000 cells. It should be appreciated that the number of cells in a multicellular aggregate (MA) may be less than about 10,000 cells. It should be appreciated that the number of cells in a multicellular aggregate (MA) may be less than about 1,000 cells. It should be appreciated that the he number of cells in a multicellular aggregate (MA) may vary as required or desired for any particular application of the method, devices, and/or deposits disclosed, discussed or referenced herein.

In describing and claiming the invention, the following terminology will be used in accordance with the following definition: articles “a” and “an” are used herein to refer to one or to more than one (i.e., to at least one) of the grammatical object of the article. By way of example, “an element” means one element or more than one element.

### Examples and Experimental Results

Practice of the invention will be still more fully understood from the following examples and experimental results, which are presented herein for illustration only and should not be construed as limiting the invention in any way. Example No. 1

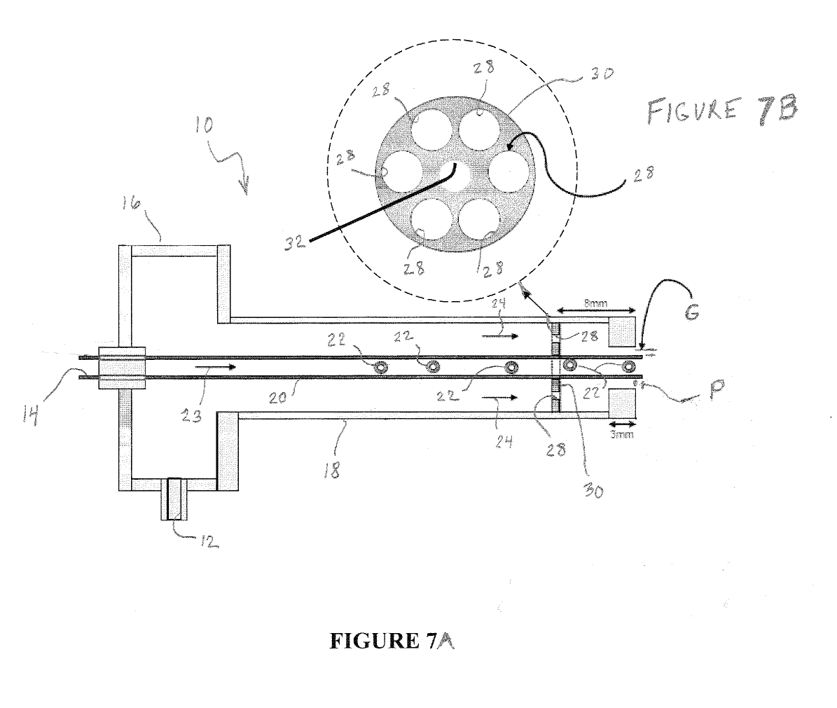

Turning to FIG. 7, FIG. 7 provides a schematic elevational view of components of a non-limiting example of the sprayer device 10. The sprayer device 10 may include the manifold 16, air port 12, multicellular aggregate port 14, outer tube 18, inner tube 20, nozzle 26, support 30, tube aperture 32, and pores 28. Also shown are the multicellular aggregates 22 in the multicellular aggregate flow 23 that may be suspended in media or fluid. The air flow 24 may travel through the pores 28 or other suitable apertures or channel. The multicellular aggregates 22 exits the nozzle where it interacts or encounters the air flow to provide the spray of the multi-cellular aggregates (MA) to be delivered to the target site (not shown).

The air flow may pass through a sterile filter (not shown) and through a rubber tubing (not shown) before entering the air flow port. The tubing may be another material or composition as desired or required. The multicellular aggregates enter the main manifold via the multicellular aggregate port and travel through the inner tube that may be a small-diameter stainless steel tube. It should be appreciated that the inner tube may be a stainless steel tube or other material or composition as desired or required. The aggregates travel down to the proximal end of the sprayer (i.e. the end that is held closest to the wound bed or tissue/organ of interest where the cell aggregates are to be delivered). The air flows around the outside of the small-diameter stainless steel inner tube down through the outer tube. As the air flow approaches the nozzle at the proximal end, it flows through pores in a stainless steel support (or other material or composition as desired or required). The air flow meets the multicellular aggregate/fluid flow at the nozzle at the proximal end. As the air flow exits the nozzle where it effectively sprays or disperses the fluid and multicellular aggregates exiting the small-diameter stainless steel tube. Although not shown a cone or other surfaces may be used to shield or redirect the flow field and thereby regulate the distance between the nozzle and the tissue bed (target) of interest.

Still referring to FIG. 7, optionally the device may comprise a proximal support 40 for the inner tube 20. The inner tube 20 may have a diameter of about 2 mm, or a dimension as desired or required. Optionally, the device may comprise a housing end cap 42 at the distal end of the device. The pores 28 optionally may be of the size of about 3.85 mm or a dimension as desired or required. The sizes of each of the pores may vary among themselves. Optionally, the inner tube may have about a 1 mm gap (or a dimension as desired or required) between the inner tube and the housing end cap, denoted as G. Optionally, the end of the inner tube may protrude about 1.5 mm (or a dimension as desired or required) beyond the end of the housing end cap, denoted a P. Optionally, the distance between the support 30 and the housing end cap may be about 8 mm (or a dimension as desired or required). Optionally, the length of the housing end cap may be about 3 mm (or a dimension as desired or required).

### REFERENCES

The following patents, applications and publications as listed below and throughout this document are hereby incorporated by reference in their entirety herein.

The devices, systems, compositions and methods of various embodiments of the invention disclosed herein may utilize aspects disclosed in the following references, applications, publications and patents and which are hereby incorporated by reference herein in their entirety:
1. Duncan C O, Shelton R M, Naysaria H, Balderson D S, Papini R P, Barralet J E. (2005) In vitro transfer of keratinocytes: Comparison of transfer from fibrin membrane and delivery by aerosol spray. J Biomed Mater Res B Appl Biomater. 73: 221-8.
2. Roberts A, Wyslouzil B E, Bonassar L. (2005) Aerosol delivery of mammalian cells for tissue engineering. Biotechnol Bioeng. 91: 801-7.
3. Falanga V, Iwamoto S, Chartier M, Yufit T, Butmarc J, Kouttab N, Shrayer D, Carson P. (2007) Autologous bone marrow-derived cultured mesenchymal stem cells delivered in a fibrin spray accelerate healing in murine and human cutaneous wounds. Tissue Eng. 13: 1299-312.
4. Waldrep, J., Berlinski, A., Dyhand, R., (2007) Comparative Analysis of Methods to Measure Aerosols Generated by a Vibrating Mesh Nebulizer. Journal of Aerosol Medicine 20: 310-319.

The following patents, applications and publications as listed below and throughout this document are hereby incorporated by reference in their entirety herein.

The devices, systems, compositions and methods of various embodiments of the invention disclosed herein may utilize aspects disclosed in the following references, applications, publications and patents and which are hereby incorporated by reference herein in their entirety:
1. U.S. Pat. No. 6,479,052, Marshall, et al., Nov. 12, 2002, “Spray Delivery of Cells”.
2. U.S. Patent Application Publication No. 2004/0219133, Lyles, Nov. 4, 2004, “Keratinocyte-Fibrocyte Concomitant Grafting for Wound Healing”.
3. U.S. Patent Application Publication No. 2007/0042488, Bornemann, Feb. 22, 2007, “Cell Spraying Device, Method and Sprayed Cell Suspension”.
4. U.S. Pat. No. 4,458,678, Yannas, et al., Jul. 10, 1984, “Cell-Seeding Procedures, Involving Fibrous Lattices”.
5. U.S. Pat. No. 5,861,149, Ritter, Jan. 19, 1999, “Methods for Wound Treatment”.
6. U.S. Patent Application No. 2007/0269498, Zhou, Nov. 22, 2007, “New Dressing Material Promoting Recovery of Skin Wound”.
7. U.S. Patent Application Publication No. 2007/0099881, Barton, et al., May 3, 2007, “Method of Treating Wounds by Enhancing Expression of Procollagen”.
8. U.S. Patent Application Publication No. 2007/0161936, Svetlik, Jul. 12, 2007, “Wound Treatment-Dressing and Method of Manufacture”.
9. U.S. Patent Application Publication No. 2003/0211137, Sierra, Nov. 13, 2003, “Foam-Forming Wound Dressing”.
10. U.S. Patent Application Publication No. 2003/0049716, Barton, et al., Mar. 13, 2003, “Method of Treating Wounds by Enhancing Expression of Precollagen”.
11. U.S. Pat. No. 5,571,079, Bello, et al., Nov. 5, 1996, “Wound Dressing”.

It should be appreciated that as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g. rat, dog, pig, monkey), etc. It should be appreciated that the subject may be any applicable human patient, for example.

In summary, the devices, systems, compositions and methods of various embodiments of the invention disclosed herein may comprise, but not limited thereto, the following: a sprayer device (and related method) for delivering (stem/therapeutic) cell aggregates into wound beds or other tissues and organs of interest. Further, embodiments of the invention sprayer and related method will be widely applicable to many wound healing, tissue repair, and regenerative applications where the delivery of cell therapy is desired.

An aspect of an embodiments of the present invention may be utilized for a number of products and services, such as but not limited thereto, the following: application of cells/aggregates into/onto wounds, organs, tissues, surfaces for therapeutic, production, manufacturing, diagnostic purposes and/or intentions.

An aspect of various embodiments of the present invention may provide a number of advantages, such as but not limited thereto, the following: efficient cell delivery; easier for end user than other delivery options; advantages for manufacturing and inventory/storage issues related to tissue engineered and cell-based products; and more cost-efficient process.

In summary, while the present invention has been described with respect to specific embodiments, many modifications, variations, alterations, substitutions, and equivalents will be apparent to those skilled in the art. The present invention is not to be limited in scope by the specific embodiment described herein. Indeed, various modifications of the present invention, in addition to those described herein, will be apparent to those of skill in the art from the foregoing description and accompanying drawings. Accordingly, the invention is to be considered as limited only by the spirit and scope of the following claims, including all modifications and equivalents.

Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Unless clearly specified to the contrary, there is no requirement for any particular described or illustrated activity or element, any particular sequence or such activities, any particular size, speed, material, dimension or frequency, or any particularly interrelationship of such elements. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all sub ranges therein. Any information in any material (e.g., a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

1. A method of delivering multicellular aggregates to a target surface of a subject, wherein said target surface comprises at least one of a wound, tissue, or organ, said method comprising:
obtaining said multicellular aggregates (MA);
suspending said multicellular aggregates (MA); and
spraying the suspension of multicellular aggregates (MA) on the target surface.

2. The method of claim 1, wherein said obtaining comprises: fabricating said multicellular aggregates by assembling cells into 3-D aggregates that have already grown in culture.

3. The method of claim 1, wherein said obtaining comprises: fabricating said multicellular aggregates by pretreating cells and/or pretreating multicellular aggregates (MA) that have already grown in culture.

4. The method of claim 1, wherein at least one of said multicellular aggregates (MA) has a cross-section of about 100 μm to about 10 mm.

5. The method of claim 1, wherein at least one of said multicellular aggregates (MA) has a cross-section of about 200 μm to about 5 mm.

6. The method of claim 1, wherein at least one of said multicellular aggregates (MA) has a cross-section of about 250 μm to about 2 mm.

7. The method of claim 1, wherein at least one of said multicellular aggregates (MA) has a cross-section of about 250 μm to about 800 μm.

8. The method of claim 1, wherein at least one of said multicellular aggregates (MA) has a cross-section of greater than about 10 mm.

9. The method of claim 1, wherein at least one of said multicellular aggregates (MA) has a cross-section of less than about 100 μm.

10. The method of claim 1, wherein said suspension of multicellular aggregates (MA) comprise human adipose-derived cells (hACs).

11. The method of claim 10, wherein said human adipose-derived cells (hACs) are autologous.

12. The method of claim 10, wherein said human adipose-derived cells (hACs) are allogeneic.

13. The method of claim 10, wherein said human adipose-derived cells (HACs) comprise: human adipose stem cells (HASCs).

14. The method of claim 10, wherein said human adipose-derived cells (HACs) comprise: human adipose progenitor cells (HAPCs).

15. The method of claim 10, wherein said human adipose-derived cells (HASCs) comprise: human adipose endothelial (HAECs).

16. The method of claim 10, wherein said human adipose-derived stem cells (HASCs) comprise: human adipose stromal cells (HAStrCs).

17. The method of claim 10, wherein said human adipose-derived cells (HACs), comprise at least one of: Human adipose stem cells (HASCs), human adipose progenitor cells (HAPCs), human adipose endothelial (HAECs), or human adipose stromal cells (HAStrCs), or any combination thereof.

18. The method of claim 1, wherein said suspension of multicellular aggregates (MA) comprise aggregates comprising at least one of: bone marrow-derived stem cells, keratinocytes, fibroblasts, hematopoietic stem cells, endothelial cell progenitor cells, perivascular (pericyte) progenitor cells, or any other type of tissue repair cell, stem cell, progenitor cell, pluripotent cell, or embryonic stem cells, or any combinations thereof.

19. The method of claim 1, wherein said suspension of multicellular aggregates (MA) are homogeneous.

20. The method of claim 1, wherein said suspension of multicellular aggregates (MA) are hybrid type.

21. The method of claim 1, wherein said suspension of multicellular aggregates (MA) comprise two or more types of aggregates.

22. The method of claim 1, wherein said wound comprises at least one of pressure ulcer, venous stasis ulcer, diabetic ulcer, burn, surgical or traumatic wound or any combination thereof.

23. The method of claim 1, wherein said tissue comprises at least one of epicardium, endocardium, vascularized tissue, dermis, epidermis, hypodermis (subcutaneous), skeletal muscle, adipose, nervous tissue, fascia, or bone, or any combination thereof.

24. The method of claim 1, wherein said organ comprises at least one of skin, heart, lung, brain, uterus, colon, eye, kidney, liver, stomach, small bowel, or pancreas.

25. The method of claim 1, wherein the number of said multicellular aggregates (MA) per unit volume of the suspension of multicellular aggregates (MA) comprises about 1 to about 10,000 multicellular aggregates per ml.

26. The method of claim 1, wherein the number of said multicellular aggregates (MA) per unit volume of the suspension of multicellular aggregates (MA) comprises about 1 to about 5,000 multicellular aggregates per ml.

27. The method of claim 1, wherein the number of said multicellular aggregates (MA) per unit volume of the suspension of multicellular aggregates (MA) comprises about 1 to about 2,000 multicellular aggregates per ml.

28. The method of claim 1, wherein the number of said multicellular aggregates (MA) per unit volume of the suspension of multicellular aggregates (MA) comprises greater than about 10,000 multicellular aggregates per ml.

29. The method of claim 1, wherein the number of said multicellular aggregates (MA) per unit volume of the suspension of multicellular aggregates (MA) comprises less than about 1 multicellular aggregate per ml.

30. The method of claim 1, wherein said multicellular aggregates (MA) suspension has a volume in the range of about 0.1 ml to about 10,000 ml.

31. The method of claim 1, wherein said multicellular aggregates (MA) suspension has a volume in the range of about 1 ml to about 5,000 ml.

32. The method of claim 1, wherein said multicellular aggregates (MA) suspension has a volume in the range of about 1 ml to about 1,000 ml.

33. The method of claim 1, wherein said multicellular aggregates (MA) suspension has a volume in the range of about 1 ml to about 500 ml.

34. The method of claim 1, wherein said multicellular aggregates (MA) suspension has a volume in the range greater than about 10,000 ml.

35. The method of claim 1, wherein said multicellular aggregates (MA) suspension has a volume in the range less than about 0.1 ml.

36. The method of claim 1, wherein said multicellular aggregates (MA) suspension spray has a flow rate in the range of about 0.05 ml/min to about 5000 ml/min.

37. The method of claim 1, wherein said multicellular aggregates (MA) suspension spray has a flow rate in the range of about 0.05 ml/min to about 1000 ml/min.

38. The method of claim 1, wherein said multicellular aggregates (MA) suspension spray has a flow rate in the range of about 0.1 ml/min to about 500 ml/min.

39. The method of claim 1, wherein said multicellular aggregates (MA) suspension spray has a flow rate greater than about 5000 ml/min.

40. The method of claim 1, wherein said multicellular aggregates (MA) suspension spray has a flow rate less than about 0.05 ml/min.

41. The method of claim 1, wherein said delivering comprises adherence of MAs to at least a portion of the target surface.

42. The method of claim 1, wherein said deposited suspension of multicellular aggregates (MA) produces a dispersed biological dressing, network, association, slurry, gel, or system of MAs, or any combination thereof on at least a portion of the target surface.

43. The method of claim 42, wherein the dispersion on said at least a portion of the target surface promotes bioactive factors.

44. The method of claim 43, wherein the bioactive factors comprises at least one of VEGF, HGF, or TGF-beta, or any combination thereof.

45. The method of claim 42, wherein the dispersion on said at least a portion of the target surface promotes matrix factors.

46. The method of claim 45, wherein the matrix factors comprise at least one of collagen, fibronectin, decorin, or tenascin C, or any combination thereof.

47. The method of claim 42, wherein the dispersion on said at least a portion of the target surface comprises cells that promote subsequent cell-cell interactions.

48. The method of claim 42, wherein the dispersion on said at least a portion of the target surface promotes bioactive factors, matrix factors, or cell-cell interactions, or any combination thereof.

49. The method of claim 1, wherein said suspension comprises a fluid or media comprising at least one of the following: biologic and synthetic biocompatible systems such as reverse-thermal gelling poloxamers (e.g. Pluronic F68, Pluronic F127), chitosan, hyaluronic acid, hydrogels, buffers, saline, thrombin/fibrin, or platelet rich plasma (PRP), or any combinations thereof.

50. The method of claim 1, wherein said suspension comprises a fluid or media comprising at least one of the following: medication, drug, coloration, dye, bioactive factor, or anesthetic, or any combination thereof.

51. The method of claim 1, wherein at least one of said multicellular aggregates (MA) have about 25,000 to about 50,000 cells therein.

52. The method of claim 1, wherein at least one of said multicellular aggregates (MA) have greater than about 50,000 cells therein.

53. The method of claim 1, wherein at least one of said multicellular aggregates (MA) have less than about 25,000 cells therein.

54. A device for delivering multicellular aggregates to a target surface of a subject, wherein said target surface comprises at least one of a wound, tissue, or organ, said device comprising:
a multicellular aggregate (MA) source;
a multicellular aggregate (MA) tube for accommodating a suspension of the multicellular aggregates (MA) traveling from the proximal end to the distal end of said multicellar aggregate tube;
an air flow tube, said air flow tube for accommodating the air flow traveling from the proximal end to the distal end of said air flow tube; and
a nozzle whereby said air flow exits said nozzle, and whereby said air flow carries the multicellular aggregates (MA) suspension for deposition on the target surface.

55. The device of claim 54, wherein said multicellular aggregates are provided by fabricating said multicellular aggregates (MA) by assembling cells into 3-D aggregates that have already grown in culture.

56. (canceled)

57. The device of claim 54, wherein at least one of said suspension of multicellular aggregates (MA) has a cross-section of about 100 μm to about 120 mm.

58. The device of claim 54, wherein at least one of said multicellular aggregates (MA) has a cross-section of about 200 μm to about 5 mm.

59. The device of claim 54, wherein at least one of said multicellular aggregates (MA) has a cross-section of about 250 μm to about 2 mm.

60. The device of claim 54, wherein at least one of said multicellular aggregates (MA) has a cross-section of about 250 μm to about 800 μm.

61. The device of claim 54, wherein at least one of said multicellular aggregates (MA) has a cross-section of greater than about 10 mm.

62. The device of claim 54, wherein at least one of said multicellular aggregates (MA) has a cross-section of less than about 100 μm.

63. The device of claim 54, wherein said suspension of multicellular aggregates (MA) comprise human adipose-derived cells (hACs).

64. The device of claim 63, wherein said human adipose-derived cells (hACs) are autologous.

65. The device of claim 63, wherein said human adipose-derived cells (hACs) are allogeneic.

66. The device of claim 63, wherein said human adipose-derived cells (HACs) comprise: human adipose stem cells (HASCs).

67. The device of claim 63, wherein said human adipose-derived cells (HACs) comprise: human adipose progenitor cells (HAPCs).

68. The device of claim 63, wherein said human adipose-derived cells (HASCs) comprise: human adipose endothelial (HAECs).

69. The device of claim 63, wherein said human adipose-derived stem cells (HASCs) comprise: human adipose stromal cells (HAStrCs).

70. The device of claim 63, wherein said human adipose-derived cells (HACs) comprise at least one of: Human adipose stem cells (HASCs), human adipose progenitor cells (HAPCs), human adipose endothelial (HAECs), or human adipose stromal cells (HAStrCs), or any combination thereof.

71. The device of claim 54, wherein said suspension of multicellular aggregates (MA) comprise aggregates comprising at least one of: bone marrow-derived stem cells, keratinocytes, fibroblasts, hematopoietic stem cells, endothelial cell progenitor cells, perivascular (pericyte) progenitor cells, or any other type of tissue repair cell, stem cell, progenitor cell, pluripotent cell, or embryonic stem cells, or any combinations thereof.

72. The device of claim 54, wherein said suspension of multicellular aggregates (MA) are homogeneous.

73. The device of claim 54, wherein said suspension of multicellular aggregates (MA) are hybrid type.

74. The device of claim 54, wherein said suspension of multicellular aggregates (MA) comprise two or more types of aggregates.

75. The device of claim 54, wherein said wound comprises at least one of pressure ulcer, venous stasis ulcer, diabetic ulcer, burn, surgical or traumatic wound, or any combination thereof.

76. The device of claim 54, wherein said tissue comprises at least one of epicardium, endocardium, vascularized tissue, dermis, epidermis, hypodermis (subcutaneous), skeletal muscle, adipose, nervous tissue, fascia, or bone, or any combination thereof.

77. The device of claim 54, wherein said organ comprises at least one of skin, heart, lung, brain, uterus, colon, eye, kidney, liver, stomach, small bowel, or pancreas.

78. The device of claim 54, wherein the number of said multicellular aggregates (MA) per unit volume of the suspension of multicellular aggregates (MA) comprises about 1 to about 10,000 multicellular aggregates per ml.

79. The device of claim 54, wherein the number of said multicellular aggregates (MA) per unit volume of the suspension of multicellular aggregates (MA) comprises about 1 to about 5,000 multicellular aggregates per ml.

80. The device of claim 54, wherein the number of said multicellular aggregates (MA) per unit volume of the suspension of multicellular aggregates (MA) comprises about 1 to about 2,000 multicellular aggregates per ml.

81. The device of claim 54, wherein the number of said multicellular aggregates (MA) per unit volume of the suspension of multicellular aggregates (MA) comprises greater than about 10,000 multicellular aggregates per ml.

82. The device of claim 54, wherein the number of said multicellular aggregates (MA) per unit volume of the suspension of multicellular aggregates (MA) comprises less than about 1 multicellular aggregate per ml.

83. The device of claim 54, wherein said multicellular aggregates (MA) suspension has a volume in the range of about 0.1 ml to about 10,000 ml.

84. The device of claim 54, wherein said multicellular aggregates (MA) suspension has a volume in the range of about 1 ml to about 5,000 ml.

85. The device of claim 54, wherein said multicellular aggregates (MA) suspension has a volume in the range of about 1 ml to about 1,000 ml.

86. The device of claim 54, wherein said multicellular aggregates (MA) suspension has a volume in the range of about 1 ml to about 500 ml.

87. The device of claim 54, wherein said multicellular aggregates (MA) suspension has a volume in the range of greater than about 10,000 ml.

88. The method of claim 54, wherein said multicellular aggregates (MA) suspension has a volume in the range less than about 0.1 ml.

89. The device of claim 54, wherein said multicellular aggregates (MA) suspension spray has a flow rate in the range of about 0.05 ml/min to about 5000 ml/min.

90. The device of claim 54, wherein said multicellular aggregates (MA) suspension spray has a flow rate in the range of about 0.05 ml/min to about 1000 ml/min.

91. The device of claim 54, wherein said multicellular aggregates (MA) suspension spray has a flow rate in the range of about 0.1 ml/min to about 500 ml/min.

92. The device of claim 54, wherein said multicellular aggregates (MA) suspension spray has a flow rate greater than about 5000 ml/min.

93. The device of claim 54, wherein said multicellular aggregates (MA) suspension spray has a flow rate less than about 0.05 ml/min.

94. The device of claim 54, wherein said nozzle is configured to adhere MAs to at least a portion of the target surface.

95. The device of claim 54, wherein said deposited suspension of multicellular aggregates (MA) produces a dispersed biological dressing, network, association, slurry, gel, or system of MAs, or any combination thereof on at least a portion of the target surface.

96. The device of claim 95, wherein the dispersion on said at least a portion of the target surface promotes bioactive factors.

97. The device of claim 96, wherein the bioactive factors comprises at least one of VEGF, HGF, or TGF-beta, or any combination thereof.

98. The device of claim 95, wherein the dispersion on said at least a portion of the target surface promotes matrix factors.

99. The device of claim 98, wherein the matrix factors comprise at least one of collagen, fibronectin, decorin, or tenascin C, or any combination thereof.

100. The device of claim 95, wherein the dispersion on said at least a portion of the target surface comprises cells that promote subsequent cell-cell interactions.

101. The device of claim 95, wherein the dispersion on said at least a portion of the target surface promotes bioactive factors, matrix factors, or cell-cell interactions, or any combination thereof.

102. The device of claim 54, wherein said suspension comprises a fluid or media comprising at least one of the following: biologic and synthetic biocompatible systems such as reverse-thermal gelling poloxamers (e.g. Pluronic F68, Pluronic F127), chitosan, hyaluronic acid, hydrogels, buffers, saline, thrombin/fibrin, or platelet rich plasma (PRP), or any combinations thereof.

103. The device of claim 54, wherein said suspension comprises a fluid or media comprising at least one of the following: medication, drug, coloration, dye, bioactive factor, or anesthetic, or any combination thereof.

104. The device of claim 54, wherein at least one of said multicellular aggregates (MA) have about 25,000 to about 50,000 cells therein.

105. The device of claim 54, wherein at least one of said multicellular aggregates (MA) have greater than about 50,000 cells therein.

106. The device of claim 54, wherein at least one of said multicellular aggregates (MA) have less than about 25,000 cells therein.

107. The method of claim 1, wherein said delivering is provided using the device of claim 54.

108. A delivered deposit on at least a portion of a target surface of a subject, wherein said target surface comprises at least one of a wound, tissues, or organs, wherein said deposit comprises a biological dressing, network, association, slurry, gel, or system of a multicellular aggregates (MA).

109. The delivered deposit of claim 108, wherein the deposit on said at least a portion of the target surface promotes bioactive factors, matrix factors, or cell-cell interactions, or any combination thereof.

110. The delivered deposit of claim 108, produced by the method of claim 1.

111. The delivered deposit of claim 108, produced by the device of claim 54.
